(19) United States
(12) Patent Application Publication
(10) Pub. No.: US 2014/0154689 A1
(43) Pub. Date: Jun. 5, 2014

(54) ANALYSIS OF CIRCULATING TUMOR CELLS AS DIAGNOSTIC AND PREDICTIVE BIOMARKERS FOR METASTATIC CANCERS

(71) Applicants: Tim Hui-Ming Huang, San Antonio, TX (US); Chun-Liang Chen, Helotes, TX (US); Joseph Liu, San Antonio, TX (US); Chiou-Miin Wang, Helotes, TX (US); Devalingam Mahalingam, San Antonio, TX (US); Pawel Osmulski, Hondo, TX (US)

(72) Inventors: Tim Hui-Ming Huang, San Antonio, TX (US); Chun-Liang Chen, Helotes, TX (US); Joseph Liu, San Antonio, TX (US); Chiou-Miin Wang, Helotes, TX (US); Devalingam Mahalingam, San Antonio, TX (US); Pawel Osmulski, Hondo, TX (US)

(21) Appl. No.: 14/098,112

(22) Filed: Dec. 5, 2013

Related U.S. Application Data
(60) Provisional application No. 61/733,746, filed on Dec. 5, 2012.

(51) Int. Cl. G01N 33/574

(52) U.S. Cl. CPC G01N 33/574
USPC 435/6.12; 435/7.23

(57) ABSTRACT

A method of analyzing circulating tumor cells includes separating circulating tumor cells from a subject's blood sample by filtration; separating CD45-negative cells from any retained blood cells; and determining the EMT-related gene expression profiles, nanomechanical and/or nanochemical properties of the collected CD45-negative CTC cells. Analysis of the collected circulating tumor cells enables the investigators to make a quick and accurate assessment of the metastatic behavior of the subject's circulating tumor cells for early castration-resistance and metastasis prediction in a clinical setting.

### PRIORITY CLAIM

This application claims priority to U.S. Provisional Application Ser. No. 61/733,746 entitled “ANALYSIS OF CIRCULATING TUMOR CELLS AS DIAGNOSTIC AND PREDICTIVE BIOMARKERS FOR METASTATIC CANCERS” filed Dec. 5, 2012.

### STATEMENT REGARDING FEDERALLY SPONSORED RESEARCH OR DEVELOPMENT

This invention was made with government support under U54CA113001 awarded by (Integrative Cancer Biology Program) of the National Institutes of Health. The government has certain rights in the invention.

### BACKGROUND OF THE INVENTION

1. Field of the Invention

The invention generally relates to the collection and analysis of circulating tumor cells.

2. Description of the Relevant Art

During the formation and growth of a prostate and other tumors, malignantly transformed cells can be shed from the primary site and circulate in the bloodstream. These circulating tumor cells (“CTCs”) are found at very low levels, one in a billion blood cells, and most die in during circulation. Nonetheless, a significant portion of these rare cells survive and can be further preprogrammed by integrins and chemokines, enabling their attachment at distant sites. After seeding to a metastatic location, CTCs adapt to survive in inhospitable conditions, e.g., low blood oxygen perfusion or low pH for extended periods. As CTCs can be obtained through routine phlebotomy, there is significant interest in their use as a measure of disease prognosis and treatment response as well as for the potential of treatment selection.

Despite the promise of CTC characterization for clinical use, detecting this rare cell population is technically challenging. The FDA-approved CellSearch® system has to date been considered the gold standard for CTC detection in the clinical setting. This system uses antibodies against the epithelial cell adhesion molecule (“EpCAM”), which positively select CTCs in a magnetic field Immunocytological analysis can then be used to confirm if these enriched cells express cytokeratins or intermediate filaments of epithelial cells, but not the common leukocyte antigen CD45. Using EpCAM-based or equivalent approaches, studies have shown that the presence of high CTC counts (≧5 cells/7.5 ml of blood) is associated with shorter progression-free survival and lower overall survival in prostate cancer patients. Furthermore, in patients with castration-resistant prostate cancer, lower CTC counts detected post-treatments can be a stronger prognostic indicator for survival.

While EpCAM-based detection technology is useful for detecting advanced prostate cancer progression, CTCs are heterogeneous and display stem cell-like properties. Emerging evidence suggests that a subset of CTCs may lack EpCAM or cytokeratin expression and instead exhibit a feature of epithelial-to-mesenchymal transition (“EMT”). EMT is a gradual process, and gene markers specific for mesenchymal and stem-like cells can be detected in CTCs. CTCs once reaching a particular site acquire an “organ-mimetic phenotype” and may lose prostate epithelial hallmarks.

### SUMMARY OF THE INVENTION

A method of analyzing circulating tumor cells includes: separating circulating tumor cells from a blood sample by filtration; separating CD45-negative circulating tumor cells from CD45-positive blood cells; and determining the nanomechanical and/or nanochemical properties of the collected CD45-negative circulating tumor cells.

In an embodiment, separating CD45-negative circulating tumor cells from CD45-positive blood cells includes: staining the collected circulating tumor cells with an anti-CD45 antibody coupled to a visualization agent; and physically separating the unstained circulating tumor cells from the stained blood cells. Physically separating the unstained circulating tumor cells from the stained blood cells includes collecting the unstained circulating tumor cells using a micromanipulator and placing the collected unstained circulating tumor cells in a container.

In one embodiment, determining the nanomechanical properties of the CD45-negative circulating tumor cells includes determining the elasticity of the CD-45 negative circulating tumor cells. Determining the elasticity of the CD-45 negative circulating tumor cells includes applying an atomic force microscope probe to at least one CD-45 negative circulating tumor cell and creating a cellular indentation mapping of the CD45-negative circulating tumor cell.

In an embodiment, determining the nanomechanical properties of the CD45-negative circulating tumor cells includes determining the smoothness of the collected CD45-negative circulating tumor cells. Determining the smoothness of the collected CD45-negative circulating tumor cells includes collecting one or more images of the collected CD45-negative circulating tumor cells and determining an average variance in height of the collected CD45-negative circulating tumor cells.

In an embodiment, determining the nanochemical features of the CD45-negative circulating tumor cells includes determining the adhesiveness of the collected CD45-negative circulating tumor cells. Determining the adhesiveness of the collected CD45-negative circulating tumor cells includes determining the force needed to pull a tip out of contact from a collected CD45-negative circulating tumor cell.

In an embodiment, the method also includes lysing the collected CD45-negative circulating tumor cells and performing single-cell RT-PCR analysis of the lysed CD45-negative circulating tumor cells to determine the expression profile of epithelial-to-mesenchymal transition (EMT) specific genes. The increased accumulative expression of EMT genes can predict castration-resistant prostate cancer and metastasis in other tumors.

In an embodiment, a method of identifying metastatic behavior of circulating tumor cells includes: separating circulating tumor cells from a blood sample by filtration; separating CD45-negative cells from residual CD45-positive blood cells retained on the filter; determining the nanomechanical and/or nanochemical properties of the collected CD45-negative cells; and determining the metastatic behavior of the collected CD45-negative cells based on the determined nanomechanical and/or nanochemical properties of the collected CD45-negative cells. In an embodiment, if a circulating tumor cell exhibits: a Young modulus of less than 1 kPa; smoothness having an RMS value of greater than 30 nm; or an adhesion of greater than 400 pN; then the circulating tumor cell is an aggressive metastatic cell.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic flow chart of CTC isolation and analyses;

FIG. 2A depicts a scheme illustrating the principle of measuring cell elasticity;

FIG. 2B shows an example of a force curve resulting from indentation of a single CTC at one preselected site;

FIG. 2C depicts a scheme illustrating the principle of measuring cell adhesion;

FIG. 2D depicts a histogram comparing elasticity of four prostate cancer cell lines;

FIG. 2E depicts a plot of RMS (nm) values (roughness) determined for different types of cells;

FIGS. 3A-C depict increased cumulative gene expression of EMT-related genes and signaling pathways in CTCs from castration-resistant patients;

FIGS. 4A-4N depict elevated expression of 14 EMT-related genes and drug target genes in metastatic prostate cancer;

FIGS. 5A-5F depict representative violin plots of EMT, drug target, stem gene panel expression profiles in four prostate cancer cells;

FIG. 6 depicts pathways shared by EMT genes upregulated and downregulated in metastatic prostate cancer cells compared to nonmetastatic benign cells.

FIGS. 7A-7C depicts box plots of Young modulus, Adhesion and Deformation of CTCs isolated from castration resistant (CR) and sensitive (CS) patients.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood that the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Described herein is an approach to enrich and process CTCs based on their unique differences in sizes and deformability that are distinct from blood and non-invasive cells. Single CTCs individually retrieved using a micromanipulator system are then subjected to atomic force microscopy (AFM) as well as microfluidics-based PCR analyses. The results show that CTCs isolated from advanced cancer patients frequently lose the typical features of epithelial cancer cells. This shift was accompanied by expressing highly diverse patterns of EMT-related genes in CTCs. Furthermore, incremental increases in the expression of these genes in these circulating cells are associated with castration-resistant and metastatic cancer.

A method of analyzing circulating tumor cells includes: separating circulating tumor cells from a blood sample by filtration; separating CD45-negative circulating tumor cells from residual CD45-positive blood cells retained on the filter; and determining the nanomechanical and/or nanochemical properties of the collected CD45-negative cells.

Circulating tumor cells may be isolated from blood samples by filtration which takes advantage of the difference in size between CTCs and blood cells. Generally CTCs are larger than blood cells, which allow the CTCs to be retained on an appropriate filter while blood cells generally pass through the filter.

To separate CD45-positive cells from the retained cells after blood filtration, the retained cells are stained with a visualization agent that causes a light detectable change in CD45-positive cells. In an embodiment, the retained cells may be reacted with an anti-CD45 antibody that is coupled to a stain (e.g., phycoerythrin). Using an appropriate visualization light source the residual CD45-positive blood cells retained on the filter can be identified and distinguished from the unstained CD45-negative cells.

The unstained CTC cells are the CD45-negative cells and can be separated from the CD45-positive cells. In one embodiment, the CD45-negative cells are separated from the CD45-positive cells by physical separation. Physically separating the unstained circulating tumor cells (i.e. the CD-45 negative cells) from the stained blood cells includes collecting the unstained circulating tumor cells using a micromanipulator and placing the collected unstained circulating tumor cells in a container. Alternatively, the stained CD45-positive cells can be removed from the collected CTC cells. Preferably, however, the unstained (CD45-negative) cells are removed. Since the method does not rely on an accurate count of the CD45-negative cells, it is only necessary to remove a statistically significant number of the unstained CD45-negative cells for nanomechanical and/or nanochemical testing.

In an embodiment, the nanomechanical and/or nanochemical properties of the separated CD45-negative cells may be used to assess the metastatic behaviors of the collected cells. Increased cell elasticity and membrane smoothness were found in metastatic CTCs compared to noncancerous cells, highlighting their potential invasiveness and mobility in the peripheral circulation. Furthermore, metastatic CTCs exhibited a higher adhesion than noncancerous cells. Despite heterogeneous expression patterns of individual CTCs, genes that promote mesenchymal transitioning into a more malignant state, including IGF1, IGF2, EGFR, FOXP3, and TGFB3, were commonly observed in these cells. It is believed that an incremental expression of epithelial-mesenchymal transition (“EMT”)-related genes in CTCs is associated with metastatic cancer. Although CTCs represent a group of highly heterogeneous cells, their unique EMT-related gene signatures provide a new opportunity for personalized treatments with targeted inhibitors in advanced prostate cancer patients.

Atomic force microscopy may be used to determine one or more nanomechanical and/or nanochemical properties of the collected CD45-negative cells. In an embodiment, individual CTCs were contacted with the cantilever of an atomic force microscope having a colloidal sphere tip (e.g., a gold colloidal sphere) with a diameter of between 1.5 to 3 micrometers. The cantilever has a nominal spring constant of between 0.01N/m and 0.1 N/m. Probes with spherical tips were used as they produce less harsh indentation than sharp tips and are less likely to cause physical damage or trigger molecular response. The surface containing the CD45-negative cells was surveyed and the probe was positioned above the selected cell. Subsequently, the height image of the cell for roughness analysis was collected in a contact mode followed by a cell indentation for the elasticity and adhesion testing.

In one embodiment, determining the nanomechanical properties of the CD45-negative circulating tumor cells includes determining the elasticity of the CD-45 negative circulating tumor cells. Determining the elasticity of the CD-45 negative circulating tumor cells includes applying an atomic force microscope probe to at least one CD-45 negative circulating tumor cell and creating a cellular indentation mapping of the CD45-negative circulating tumor cell. To determine the Young modulus, cellular indentation mapping may be performed with the AFM force spectroscopy. In some embodiments, the central area on a cell surface was probed to obtain the most consistent elasticity data. For each evaluated point, the force versus indentation curve was constructed based on the force-load plots. The Hertz model may be applied to calculate the Young's modulus using the force-indentation curves. The model describes the physical relationship between the applied force and the cantilever indentation. It assumes spherical shape of the end of a tip placed on a flat surface. The model is valid when the sphere radius is substantially larger than indentations. The elasticity for each cell was averaged, and nominal elasticity was tested against cleaned glass disks.

In an embodiment, determining the nanomechanical properties of the CD45-negative circulating tumor cells includes determining the smoothness of the collected CD45-negative circulating tumor cells. A contact mode image of each cell was collected using a scan size from 5×5 to 30×30 micrometers with a matrix of 512×512 pixels per scan at 1 Hz scan rate. We analyzed smoothness values within 2 to 4 square areas of a cell surface covering from 1 to 25 μm². As a measure of a cell membrane smoothness, the Root Mean Squared (RMS) of height calculated from heights of all image pixels included in the area of interest may be used. Images of a glass surface surrounding the cells were used as a blank.

In an embodiment, determining the nanochemical properties of the CD45-negative circulating tumor cells includes determining the adhesiveness of the collected CD45-negative circulating tumor cells. Determining the adhesiveness of the collected CD45-negative circulating tumor cells includes determining the force needed to pull a tip out of contact from a collected CD45-negative circulating tumor cell. Typically adhesiveness is measured as the tip is removed from the cell after the elasticity measurement is complete.

The nanomechanical and/or nanochemical properties of non-metastatic cells compared to metastatic cells is substantially different. Metastatic cells exhibit a significantly higher elasticity compared with non-metastatic cells. For example, cells having a Young modulus of between 1.5 kPa and 0.1 kPa exhibited some degree of metastatic behavior. Cells having a Young modulus of less than 1 kPa were determined to be aggressively metastatic.

Metastatic cells also exhibit a rougher cell surface compared to non-metastatic cells. For example, cells having a RMS height of between 25 nm and 100 nm exhibited some degree of metastatic behavior. Cells having an RMS height of greater than 30 nm were determined to be aggressively metastatic.

Metastatic cells also exhibit a greater adhesion compared to non-metastatic cells. For example, cells having an adhesion ranging from 100 pN to 2000 pN exhibited some degree of metastatic behavior. Cells having an adhesion of greater than 400 pN were determined to be aggressively metastatic.

Further tests of the isolated CD45-negative cells may be performed to determine the specific genes being expressed by the cell. In an embodiment, the method also includes lysing the collected CD45-negative circulating tumor cells and performing RT-PCR analysis of the lysed CD45-negative circulating tumor cells to determine the presence of metastatic specific genes. Standard RT-PCR analysis can be performed. For example, PCR primers of selected genes for expression profiling may be selected from the PrimerBank database. Reverse transcription preamplification, and PCR amplification is then carried out according to the protocol of single-cell gene expression. The relative gene expression of the genes of interested may be determined using any known technique (e.g., using a chip assay).

The current EpCAM-based technologies are largely restricted to count increased numbers of CTCs known to correlate with advanced cancer. Using the strategy of coupling a microfiltration system with a micromanipulator device, we have developed a new system to characterize physical properties and expression patterns of individual CTCs in advanced cancer patients, as well as in established cancer cell lines. This novel technology has permitted us to make the unexpected discovery that the majority of EpCAM-positive CTCs show loss of epithelial characteristics. Shed from the primary sites, these cells become highly deformed by increasing their membranous elasticity and smoothness. It is theorized that aberrant expression of EMT-related genes can completely or partially replace prostate epithelial features, instead displaying mesenchymal and stem-like characteristics. Activation of TGF-β signaling leads to increased activities of transcription factors in the TWIST, ZEB and SNAIL gene families that repress epithelial cell adhesion and induce other mesenchymal proteins. Overexpression of WNT agonists, FZD7 and FZD4, results in increased expression of MMP gene families that promote metastatic dissemination.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

### Isolation of Single Circulating Tumor Cells (CTCs) Using Size-Based Filtration

The University of Texas Health Science Center at San Antonio's Institutional Review Board approved the study and consent was obtained prior to sample collection. Patient blood samples (˜10 ml) were collected in K₂-EDTA tubes, which were inverted five times and kept at 4° C. or on ice. The patient blood was subjected to single CTC isolation. CTCs were first isolated from blood cells using ScreenCell® CC filtration kit (cat # CC 3LC-ha, ScreenCell, Paris, France) according to manufacturer's protocol with modifications. After blood filtration, the circular-filter was released onto an uncoated sterile petri dish with the cell-retained side up. From this point on, the rest of isolation process was carried out under an inverted Evos fl digital fluorescence microscope (cat #1253460, AMG, Bothell, Wash.). The filter was washed 2 to 3 times with 50 μL PBS. During the washes, the residual blood cells were further carried through the filter using gentle pipetting or dragging the filter against the bottom of petri dish using sterile forceps. If blood cell clumping occurred that could interfere with single CTC isolation, clumps were dissociated by incubation with 50 μL TrypLE Express (cat #12604-013, Invitrogen, Carlsbad, Calif.) for 10 min in a petri dish before PBS washes. CTCs and residual blood cells retained on the filter were stained with anti-CD45 conjugated with phycoerythrin (PE) (BD, Maryland) for 15 min and subjected to three PBS washes as described above. CTCs on the filter were incubated with 25 μL TrypLE Express for 10 min and removed and placed onto a new petri dish for CD45-negative selection and single CTC isolation using a Narishige micromanipulator and Ferty Syringe Plus Microinjector (cat # MN-153 and INJ-FS-PLUS, Origio MidAtlantic Devices, Mt. Laurel, N.J.). Single CD45-negative CTCs were isolated individually, ejected in 4.5 μL PBS with 0.5 μL lysis buffer (cat #55827, Invitrogen, Carlsbad, Calif.) in a 0.2 ml PCR tubes and frozen on dry ice immediately and stored at −20° C. until microfluidics-based PCR analysis. Some CTCs were pooled together in RPMI medium supplemented with 10% FBS and ampicillin/streptomycin for atomic force microscope analysis.

### Prostate Cell Culture

Prostate cancer cell lines, LNCaP-AD (androgen-dependent), LNCaP-AI (androgen-independent) were routinely maintained in the laboratory. BPH1, PC-3, and DU145 cell lines were obtained from ATCC. The cells were cultured in RPMI medium with 10% FBS.

### Analysis of CTCs and Prostate Cells Using Atomic Force Microscopy (AFM)

Individual CTCs and prostate cells suspended in ˜50 μl PBS were loaded on a poly-Lys (300 kD; 01% in PBS) coated glass disc glued to a steel disc. The discs were mounted in the MultiMode Nanoscope Ma microscope (Bruker) equipped with the J type scanner and the glass chamber for in-liquid work. The SQube probes with a colloidal gold sphere with a diameter of between 1.5 to 3 micrometers as a tip, and nominal spring constant of 0.08 N/m were applied for elasticity testing and topography imaging. Probes with spherical tips were used as they produce less harsh indentation than sharp tips and are less likely to cause physical damage or trigger molecular response. The surface of the glass disc was surveyed for the presence of cells under a video camera used for probe position control, and the probe was directed above the selected cell. Subsequently, the height image of the cell for roughness analysis was collected in a contact mode followed by a cell indentation for the elasticity testing. A standard plane fit was executed on the height mode images with the Nanoscope software version 5.12. Roughness and force plots were analyzed with the SPIP v.5.11 software (Image Metrology, Denmark).

Cell Elasticity.

To determine the Young modulus, we performed cellular indentation mapping with the force AFM. The central area on a cell surface was probed to obtain the most consistent elasticity data. We collected a 3×3 array of force curves (total 9 data points) covering area of 4 μm², with at least 5 indentations for each point. Indentation depth was restricted to 400 nm. A constant pulling rate was maintained throughout all the experiments. The applied design allowed for data collection in less than 2 min per cell minimizing the cell stress response induced by the prolong instrumentation of the cell surface. For each evaluated point, the force versus indentation curve was constructed based on the force-load plots. We then applied the Hertz model to calculate the Young's modulus using the force-indentation curves. The model describes the physical relationship between the applied force and the cantilever indentation. It assumes spherical shape of the end of a tip placed on a flat surface. The model is valid when the sphere radius is substantially larger than indentations. The elasticity for each cell was averaged, and nominal elasticity was tested against cleaned glass disks.

Cell Roughness.

To assess a level of nanomechanical complexity of cell membranes, we determined their surface roughness. A contact mode image of each cell was collected using a scan size from 5×5 to 30×30 micrometers with a matrix of 512×512 pixels per scan at 1 Hz scan rate. We analyzed roughness values within 2 to 4 square areas of a cell surface covering from 1 to 25 μm². When analyzing multiple patient-derived samples, we used the same spherical probe for force plots and image collecting. As a measure of a cell membrane roughness, we employed Root Mean Squared (RMS) of height calculated from heights of all image pixels included in the area of interest. Images of a glass surface surrounding the cells were used as a blank.

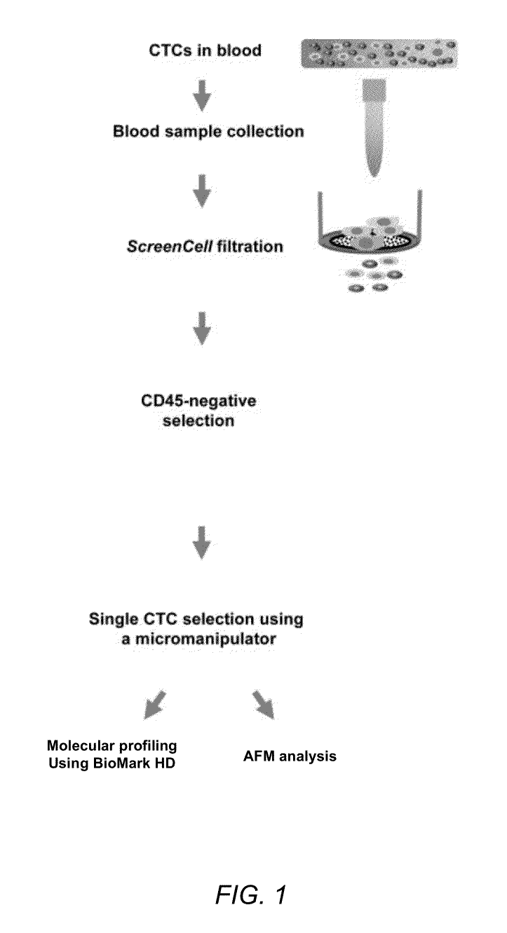

The protocol of Atomic Force Microscope Analysis:
1. PeakForce Quantitative Nanomechanical Mapping (QNM) AFM (Bruker) is applied to image CTCs obtained from peripheral blood of prostate cancer patients and cultured prostate cancer cells.
2. The cells captured on a filter are imaged directly on a filter without any additional processing: in particular they are not immobilized with any method.
3. The filter is mounted on a bottom of a 55 mm Petri dish and kept covered except during AFM imaging.
4. The cells after their capture on the filter are overlaid with 10 μL of DMEM medium and then are left for 30 min at RT. Additional 10 μL of the medium is added after each hr of imaging or sooner if needed.
5. Cells can be observed for about 3 hrs when mounted on the filter without signs of loss of their viability.
6. The filter can be stored at 4° C. for 12 hrs and then cells imaged, but certain types of cells can be affected.
7. DMEM medium can be substituted with other suitable media. PBS is not recommended because of a risk of cell starvation.
8. Cells are imaged using a Nanoscope Catalyst (Bruker) AFM mounted on a Nikon Ti inverted epifluorescent microscope.
9. SCANASYST-AIR (Bruker) tips are used for imaging.
10. The nanomechanical data are captured in the form of a cell map (PFC files: Bruker) or single point measurements (HSDC, Bruker).
11. Cell mapping allows capturing and analyzing the following data: cell elasticity, adhesion, deformation, energy dissipation, surface roughness, and topography.
12. Single point measurements allow capturing and analyzing the following data: cell elasticity, adhesion, deformation, and energy dissipation.
13. Mapping of elasticity is performed based on the Sneddon model that approximates mechanics of conical tip interactions with an object.
14. Adhesion forces are included in the analysis.
15. Single point measurements of elasticity are fitted to the Hertzian model of the spherical tip interactions included in the Bruker Nanoscope Analysis. Then the obtained values are recalculated into the Sneddon model.
16. Nanomechanical parameters are calculated with Nanoscope Analysis software.
17. Roughness calculations are performed with Nanoscope Analysis (Bruker) and SPIP v. 6 (Image Metrology) software. Version 1.4 of Nanoscope allows only a square area to select as a region of interest.
18. About 143 data points were collected with the PF QNM AFM on a filter including complete images of 26 distinct cells (pfc images) and 78 hsdc plots.
19. About 75% of cells were classified as soft: their Young Modulus which constitutes a measure of elasticity was below 4 kPa. 15% of cells were classified as medium soft (4-10 KPa). 10% of cells were hard (>10 kPa). These cells did not have detectable prostate cancer markers and were excluded from further analysis
20. Adhesiveness values were between 10 and 4000 pNewtons: with poorly sticking (<100 pN) 45%, medium adherent 35% (100-399 pN), strongly adherent (400-1000 pN) 13%, very strongly adherent (>1 nN) 7%. Adhesion represents in this case force needed to pull a tip out of contact from a cell when the tip retracts (is lifted).
21. 25% of objects were poorly deformable (<100 nm), almost 28% were deformable (100-399 nm), 25% were strongly deformable (400-799 nm) and 23% were exceptionally deformable (>800 nm). Deformation of an object is defined here as a depth into which a tip travel at constant force without damaging a cell.
   FIG. 1 depicts a schematic flow chart of CTC isolation and analyses.

### Single-Cell Microfluidics-Based RT-PCR Analysis

Single-cell microfluidics-based RT-PCR analysis was carried out using CellsDirect™ one-step qRT-PCR kit (cat #11753-100, Invitrogen, Carlsbad, Calif.) and a microfluidics device, BioMark HD MX/HX system (cat # BMKHD-PKG-MH, Fluidigm, Inc., South San Francisco, Calif.) [20]. Single CTCs in PBS/lysis buffer were thawed, mixed well and spun down before lysed at 75° C. for 10 min. To reduce contamination, genomic DNA was degraded in an 18 μl reaction volume using DNase I (5 units) with 1× DNase I buffer at RT for 5 min. PCR primers of selected genes for expression profiling were selected from the PrimerBank database. These primers were divided into two panels to fit BioMark 48×48 chips.

Reverse transcription (RT), preamplification, and PCR amplification were carried out according to the protocol of single-cell gene expression (cat # BMK-M-48.48, Fluidigm). Target genes were amplified using BioMark HB MX/HX system with 1× SsoFast EvaGreen supermix with low ROX (cat # PN172-5211, Bio-Rad, Hercules, Calif.) and 1× DNA binding dye sample loading reagent (cat # PN 100-3738, Fluidigm). In each chip assay, universal RNA (200 pg) from human normal tissues (cat #4234565, BioChain, Newark, Calif.) and no template control (NTC) served as positive and negative controls.

### Data Analysis

Expression data of genes of interest were displayed in cycles of threshold (Cts) after analysis using Real-Time PCR analysis software (Fluidigm). Relative expression values of the genes was obtained using 2^{-ΔΔCt} method in that each gene expression is normalized to a reference gene and then normalized to lowest expressed genes that have Ct 40. Although three housekeeping genes (ACTB, GAPDH, and UBB) were initially included as reference genes, we found Ubiquitin B (UBB) to be a highly stable gene for microfluidics-based PCR analysis, as its reliability has previously been validated in a meta-analysis of over 1000 clinical samples. However, expression levels of ACTB and GAPDH were less stable and weaker among different CTCs, consistent with a previous finding for single-cell CTC analysis. Therefore, we only selected cells that expressed UBB at a threshold of Ct≦30 after pre-amplification, assuming that CTCs expressing robust expression of UBB are less likely to contain degraded RNA. Loge values of gene expression in each CTC were summed up as cumulative gene expression according to the groups of frequently expressed EMT-related genes (detected in >44% CTCs) and less frequently expressed EMT-related genes (present in <44% CTCs) and different oncogenic signaling pathways for comparisons. Cumulative gene expressions of CTCs from prostate cancer patients were analyzed using one-way ANOVA and unpaired Student's t test using Prism 6 (GraphPad Software, La Jolla, Calif.). A p value of <0.05 is considered as statistically significant.

For in silico analysis of EMT-related gene expression in clinical samples, raw probe cel intensity (*.cel) files were obtained from Gene Expression Omnibus (GEO) series GSE6919. Expression data for samples representing Normal Prostate Tissue free of any pathological alteration (n=18), Normal Prostate Tissue Adjacent to Tumor (1=63), Primary Prostate Tumor (n=65) and Metastatic Prostate Tumor (n=25), generated using Affymetrix Human Genome U95 Version 2 Array were used for this study. RMA (Robust Multichip Average) expression measures were calculated for probes in all the samples by RMA normalization and background correction using Bioconductor Affy package in R. The expression was then collapsed to gene level by averaging the measures for the probes representing a gene. These expression data were further used to compare the Metastatic samples with Normal samples by calculating the significance (using Student's t-test along with Benjamini, Hochberg false discovery rate adjustment) and fold change.

### Results

### Increased Elasticity and Smoothness of Cell Surface Membrane in CTCs

The separation of malignant cells from the primary site via acquisition of invasive properties and transport into the bloodstream are initial steps of metastasis. To characterize CTCs, we used a microporous device to filter and select CD45-negative cells from blood samples (Table 1). Larger than blood cells, these cells showed irregular fibroblastoid morphology, suggestive of epithelial to mesenchymal transition.

TABLE 1
Clinical Information of Prostate Cancer Patients

| | | Gleason | PSA | CTC Isolated | | |
|---|---|---|---|---|---|---|
| Patient | Age | Score | (ng/ml) | (# analyzed) | Metastasis | Status of Treatment |
| CC01 | 72 | 9 | 331.47 | 84 (10) | bone | castration-resistant |
| | | | | | | chemo-resistant |
| CC02 | 61 | 9 | 88.47 | 9 (6) | bone | castration-resistant |
| CC03 | 71 | 9 | 96.05 | 6 (4) | bone | castration-resistant |
| CC04 | 53 | 9 | 79.00 | 4 (2) | bone (small volume) | castration-resistant |
| | | | | | lymph nodes | chemo-resistant |
| CC06 | 80 | 9 | 6.4 | 44 (5) | bone (small volume) | castration-resistant |
| | | | | | | immunotherapy-responsive |
| CC07 | 62 | 9 | 1191.56 | 151 (6) | bone (small volume) | castration-sensitive |
| | | | | | lymph nodes | |
| CC08 | 60 | N/A | 13.58 | 6 (3) | bone | castration-sensitive |
| | | | | | lymph nodes | |
| CC09 | 64 | 8 | 0.26 | 4 (2) | none | castration-sensitive |

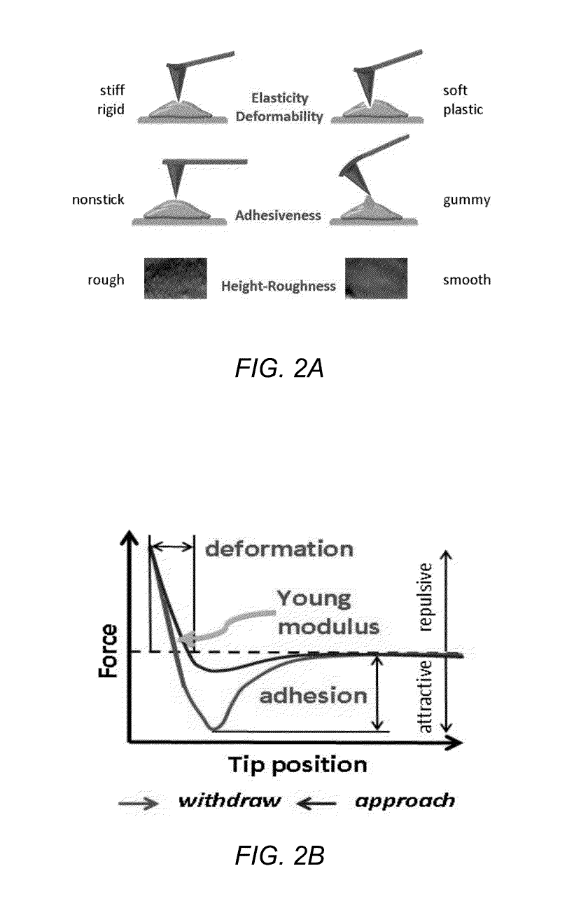

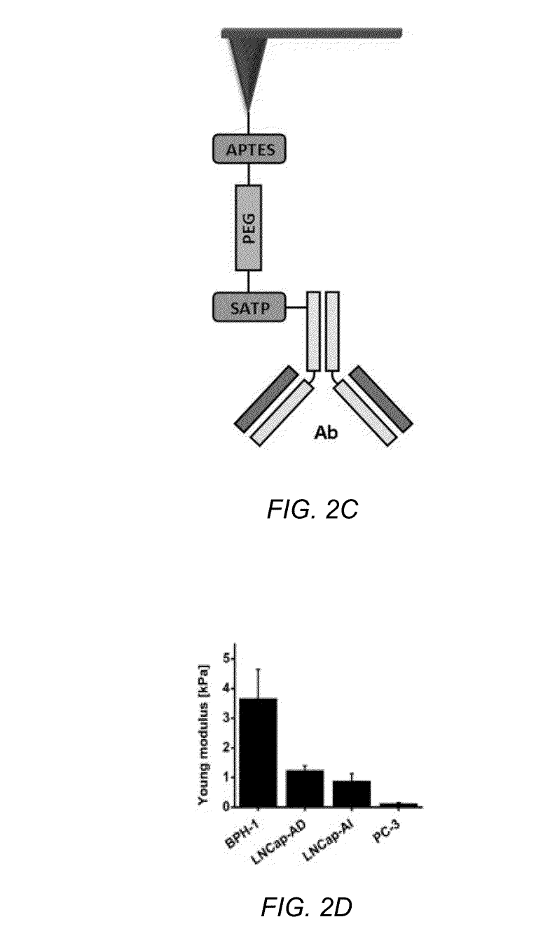

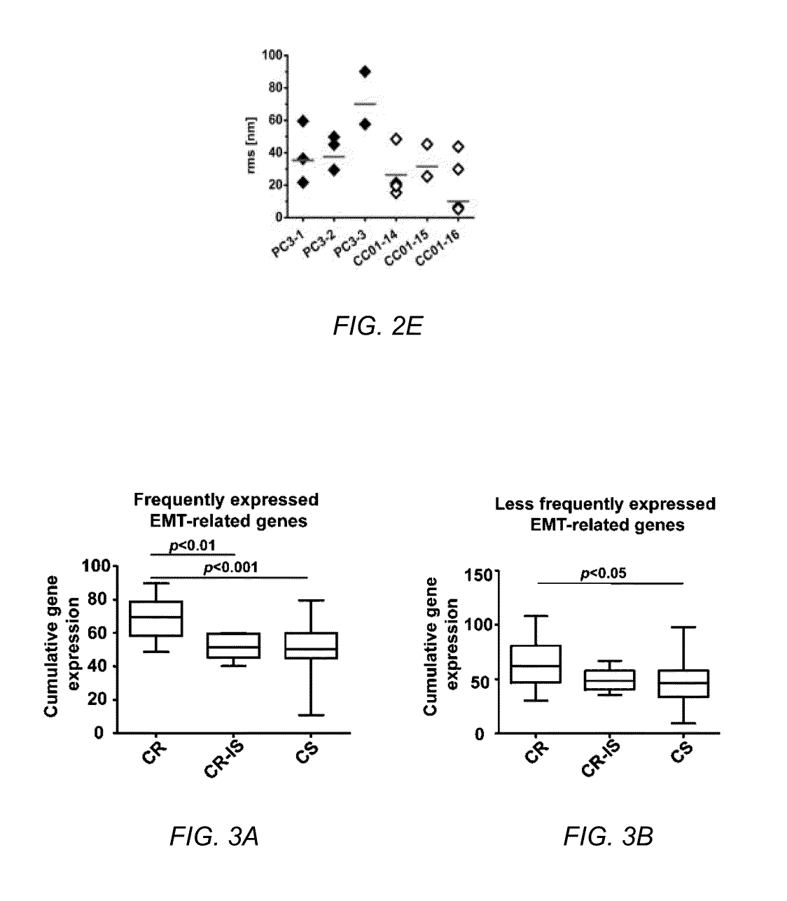

The CTCs were individually retrieved by a micromanipulator and used to determine their surface topography and mechanical properties by AFM. The AFM-based analysis utilizes interactions between a probe (“tip”) and a cell. Raster scanning of the cell with a probe results in the image of cell surface, suitable for comparing general features of surface topography, here represented by membrane roughness, between individual single cells. On the other hand, in the AFM force mode the probe indents a cell with a controlled force load. As a result, the cantilever to which the probe is attached is deflected proportionally to the applied force. FIG. 2A depicts a scheme illustrating the principle of measuring cell elasticity. A cell (blue) bound to a mica surface (grey) is indented by a tip (red triangle) mounted on a flexible cantilever (red board) proportionally to the cell elasticity. Deflection of the cantilever (blue arrow) changes a position of a laser beam reflection that measures force needed to indent the cell. The distance between a tip end and the cell is represented by the Z position (thick vertical arrow) directly measured by a piezoelectric element of the microscope.

FIG. 2B shows an example of a force curve resulting from indentation of a single CTC at one preselected site. A blue trace represents a tip approach phase in which the tip is brought into a direct contact with a cell surface from approximately 1000 to 700 nm. Next, the cantilever is progressively deflected as the tip encounters stronger cell resistance. At a preset Z position, a tip stops and then retracts (red trace) not exactly following the approach trace. Blue arrows point at positions of little humps at which the tip likely sensed a cytoskeleton discontinuity. Adhesion forces between the tip and the cell bent the cantilever in the opposite direction as indicated by the red arrow.

FIG. 2C depicts a histogram comparing elasticity of four prostate cell lines. Histograms represent mean values with corresponding SD. Based on a plot describing dependence of the cantilever deflection on indentation, the Young modulus constituting a measure of individual cell elasticity was derived. We determined the Young modulus of the cultured cells from the following established lines: the immortalized BPH-1 prostate cells and three prostate cancer cell lines, LNCap-AD, LNCap-AI, and PC-3. Noncancerous BPH-1 cells were the least elastic with the Young modulus about 3.7-kilopascal (kPa), whereas the highly metastatic PC-3 cells were almost 30× more elastic (0.13-kPa. Interestingly, androgen-independent LNCap-AD cells were more elastic then androgen-dependent LNCap-A1 (0.88-kPa versus 1.2-kPa).

FIG. 2D depicts histograms comparing elasticity of four CTCs isolated from blood of a patient with castrate-resistant prostate cancer and bone metastasis. Young moduli of these CTCs ranged from 0.23-kPa to 1.1-kPa, and the obtained values were similar to that of PC-3 elasticity, but much lower than those values calculated for BPH-1 cells.

To determine cell surface roughness, images of the same cells were acquired immediately after elasticity determination, by scanning the cells in contact mode with the same spherical probe. We measured roughness with a root RMS parameter, which corresponds to a variance of pixel heights included in an area of interest. The RMS is measured in nm and does not depend on area size in the range of 1 to 5 μm². Therefore, the higher RMS value reflects a more rich relief of a cell surface and its lower value corresponds to a smoother surface. RMS values found in a single cell were quite diverse reaching from 22 to 90 nm. FIG. 2E depicts a plot of RMS (nm) values determined for different types of cells. On average the PC-3 cells showed a rougher cell surface than CTCs, which appeared smoother. Specifically, the average RMS for all the PC-3 cells was 48.7 nm, whereas for CTCs was only 25.2 nm with the difference statistically significant at p<0.05.

The AFM analysis presented here indicates that cell elasticity and smoothness can be considered useful parameters to distinguish between non-metastatic and metastatic cells. Differences in elasticity also reflect a histological background of a cell. The smoothness, commonly used to characterize a surface property of a variety of materials, reflects cell mobility, distribution of surface proteins, and loss of cell polarity. These results suggest that the high deformity and high smoothness of CTC membrane surface can be the result of a morphological transitioning of these cells into mesenchymal-like cells for malignant invasion. Considering the changes in a cell membrane accompanying EMT and propensity to adhere, we expect that softer and smoother cells represent the most aggressive metastatic cells possibly indicating poor prognosis.

### Loss of Epithelial Prostate Cancer Features in CTCs

Our microporous filtration-micromanipulator system was further used to isolate 308 CD45-negative CTCs from blood samples of 8 prostate cancer patients (Table 1). CTCs were not detectable in blood samples from two healthy individuals (data not shown). Sixty-two of these captured cells were subjected to single-cell microfluidics-based RT-PCR analysis of a panel of 11 known prostate epithelial markers and two control genes. The 11 prostate epithelial markers were: EpCAM; ACPP (PSAP); MK167 (Ki-67); KLK4; PCA-3; FOLH1 (PSMA); KLK2; KRT5 (CK5); KRT7 (CK7); KRT8 (CK8); and PSA (KLK3). The two controls were PTPRC (CD45) and UBB. Of these, 38 cells showed robust expression of UBB, and their expression data were subsequently used for normalization with the expression value of this housekeeping gene. Included in the analysis were three prostate cancer cell lines—PC-3, DU145, and LNCap-AD and universal RNA as a positive control and water as a negative control.

A heat map of the panel analysis displays a remarkable heterogeneity of gene expression in the 38 CTCs analyzed. The majority (93%) of these cells expressed EpCAM, suggesting their epithelial origin. However, only ˜20% of these CTCs showed detectable PSA and PCA-3 that are known to encode common prostate-specific antigens. Other prostate cancer markers (e.g., PSAP and PSMA) and epithelial markers (CK5, CK7, and CK8) were also present in 20% of these circulating cells. Seven cells were EpCAM-negative, but expressed various prostate-related gene markers. Although we cannot rule out technical limitations of detecting some prostate cancer-related genes at the single-cell level, our initial results suggest a dramatic shift of gene expression occurring in CTCs that escaped from their primary tumor sites. When seeded in metastatic locations, these cells may recirculate back into the bloodstream and progressively lose their epithelial prostate characteristics.

### Cumulative Expression of EMT-Related Genes in CTCs of Castration-Resistant Cancer

Because of the invasive nature of CTCs, we also determined expression profiles of 49 EMT-related genes in these prostate cancer patients that were categorized into castration-resistant (i.e., four patients resistant to both castration therapy and docetaxel chemotherapy), one castration-resistant/immunotherapy-responsive (in regards to patient's serum PSA response observed following the Provenge immunotherapy), and castration-sensitive (i.e., three patients obtaining PSA response following initiation of castration therapy) groups (see Table 1). The 49 EMT-related genes investigated were: PTPRN2; ALDH1(A1); CXCL13; ESR2 (ESRb); ASPA; CDH2; CDH1; COL1A2; DAB2IP; FN1; VIM; ITGB1 (CD29); IGF1; IGF2; WNT5A; IGF1R; FZD4; WNT11; MMP14; MMP9; WNT5B; SNAI2; GSK3B; MMP2; MMP7; NOTCH1; SOX9; TCF3; CTNNBI; FZD7; ITGA6; PTCH1; GLI-3; PTCH2; SHH; FOXP3; TGFB3; SMAD2; TWIST1; ZEB1; BMP7; TGFB2; ZEB2; FOXC2; FOXA2; TGFB1; EGFR (Her/ERBB1); ERBB2; and SRC.

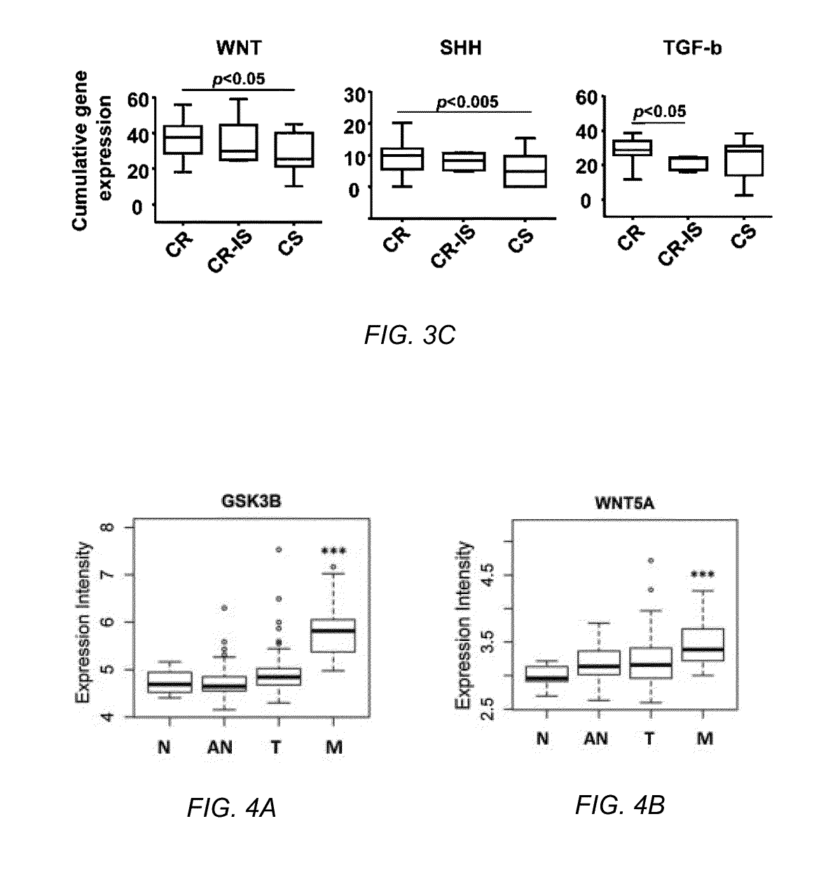

FIGS. 3A-C depict cumulative gene cumulative expression of EMT-related genes and signaling pathways in CTCs from castration-resistant patients. Cumulative expression EMT-related genes in each CTC are displayed in box plots among CR: castration-resistant; CR-IS: castration-resistant and immunotherapy sensitive; and CS: castration-sensitive patients. FIG. 3A depicts cumulative gene expression of frequently expressed EMT-related genes. FIG. 3B depicts cumulative gene expression of less frequently expression EMT-related genes. FIG. 3C depicts cumulative gene expressions of WNT, SHH and TGF-β signaling pathways. Data were analyzed using one-way ANOVA and unpaired Student's t test. A p value of <0.05 is considered as statistically significant.

Despite high degrees of transcriptional heterogeneity, 18 of these EMT-related genes were commonly expressed in 44-100% of these CTCs analyzed. Furthermore, expression levels of some of these genes (e.g., PTPRN2, ALDH1, ESR2, and WNT5A) were significantly higher in CTCs of castration-resistant patients than those of castration-resistant/immunotherapy-responsive (p<0.01) and castration-sensitive (p<0.001) patients (FIG. 3A). The expression of the remaining 24 EMT-related genes was less frequent (<44%) in these CTCs by the microfluidics-based PCR system. When expressed, incremental numbers and high expression values of these genes were significantly found in circulating cells isolated from castration-resistant patients (p<0.05) (FIG. 3B). When further categorizing EMT-related genes into different oncogenic signaling pathways, we found that upregulation of these genes was significantly associated with Sonic Hedgehog (p<0.005), WNT (p<0.05), and TGF-β (p<0.05), suggesting their important roles in metastatic castration-resistance and immunotherapy.

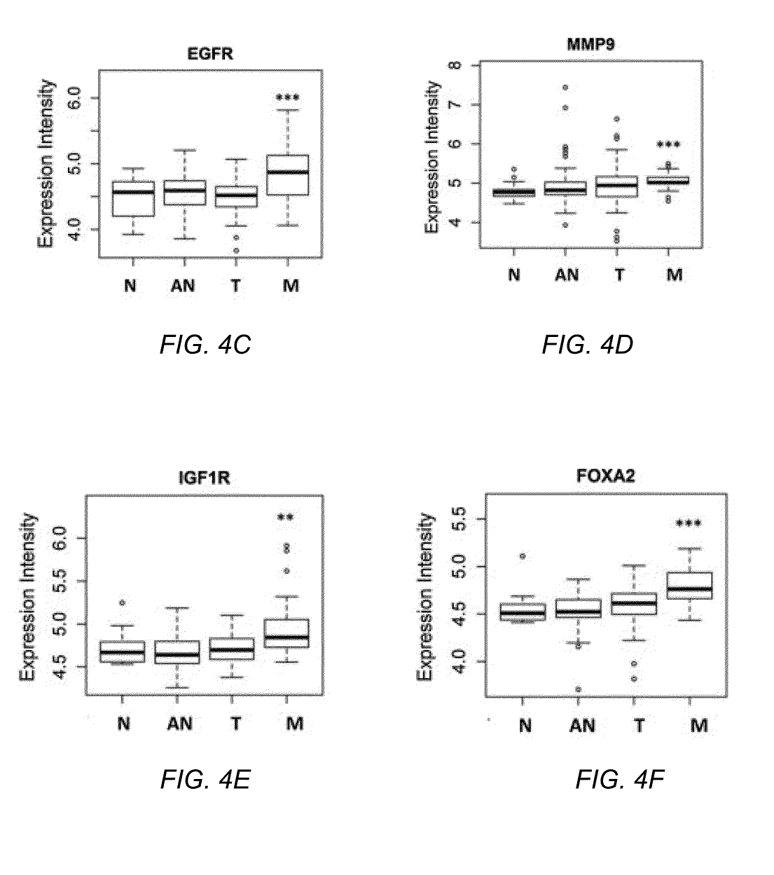

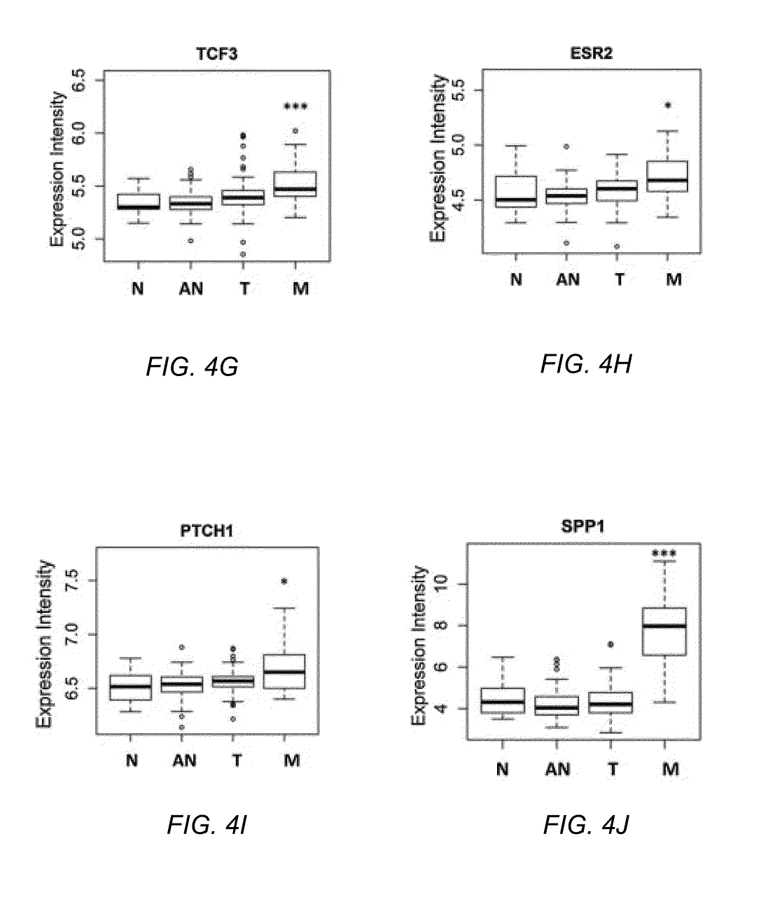

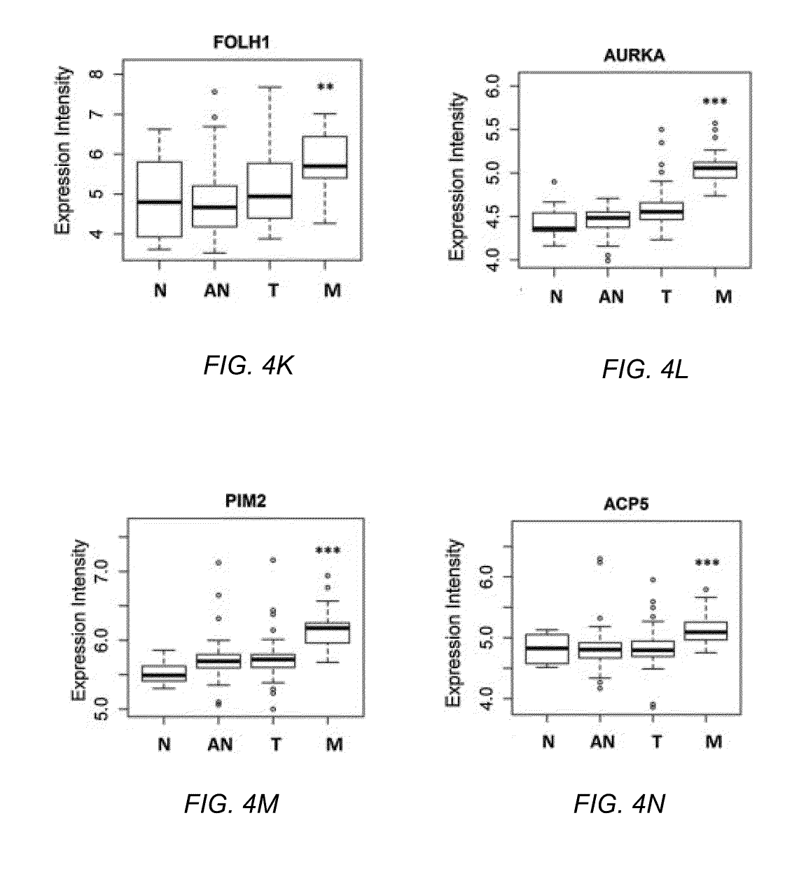

FIGS. 4A-4N depict elevated expression of 14 EMT-related genes and drug target genes in metastatic prostate cancer (GSK3B; WNT5A; EGFR; MMP9; IGF1R; FOXA2; TCF3; ESR2; PTCH1; SPP1; FOLH1; AURKA; PIM2; and ACP5, respectively). In silico analysis of gene expression revealed that expression of nine EMT-related genes and five drug target genes are higher in clinical metastatic prostate tumors than normal prostate. Data were analyzed using Student's t test. N: normal; AN: normal tissue adjacent to tumor; T: tumor; and M: metastatic. *, p<0.05; **, p<0.01; ***, p<0.001.

In silico analysis using available expression microarray data of a published prostate cancer cohort confirmed frequent upregulation of fourteen (e.g., ESR2, WNT5A, IGF1R, PTCH1, GSK3B, MMP3, PTPRC, and EGFR) of these candidate genes in metastatic sites of prostate cancer (FIG. 4A-4N).

Genes encoding for the regulation and maintenance of stem-cell characteristics were detected in CTCs, but appeared as a less frequent event (˜10%). However, two additional stem-cell gene markers, PTPRN2 and ALDH1, were related to EMT and were frequently expressed in CTCs of castration-resistant patients.

### Discussion

The current EpCAM-based technologies are largely restricted to count increased numbers of CTCs known to correlate with advanced prostate cancer. Using an innovative strategy by coupling a microfiltration system with a micromanipulator device, we have developed a new system to characterize physical properties and expression patterns of individual CTCs in advanced prostate cancer patients, as well as in established prostate cancer cell lines. This novel technology has permitted us to make the unexpected discovery that the majority of EpCAM-positive CTCs show loss of epithelial characteristics. In spite of high PSA values detected in the blood of these patients, these cells may not express PSA and other frequently detectable markers in primary prostate tumors. Shedding from the primary sites, these cells become highly deformed by increasing their membranous elasticity and smoothness. It is possible that aberrant expression of EMT-related genes can completely or partially replace prostate epithelial features, instead displaying mesenchymal and stem-like characteristics. Activation of TGF-β signaling leads to increased activities of transcription factors in the TWIST, ZEB and SNAIL gene families that repress epithelial cell adhesion and induce other mesenchymal proteins. Overexpression of WNT agonists, FZD7 and FZD4, results in increased expression of MMP gene families that promote metastatic dissemination.

Clinically relevant to this discussion is the difference observed in the EMT-related gene profiles between the patients with advanced castrate-sensitive prostate cancer (i.e., responding to castration with PSA response) and patients who are castrate- and chemo-resistant (i.e., progressed on both castration therapy and docetaxel chemotherapy). Patients with newly diagnosed advanced prostate cancer are almost always treated with medical castration therapy, the majority of which will respond favorably to therapy with improvement in PSA response, defined as a PSA≦4 ng/ml at 7 months after therapy, with those achieving a PSA≦0.2 ng/ml having a much better median overall survival of 75 months. About a third of men however fail to achieve a PSA≦4 ng/ml, develop early castrate-resistant disease, and have a median OS of just 13 months. Identifying this subset of patients early in their course of castration therapy based on expression patterns of EMT-related genes in CTCs would have prognostic value.

A further interesting finding relates to the EMT-related gene profile for one patient with castrate-resistant disease treated with the Provenge immunotherapy instead of docetaxel chemotherapy. Provenge is now being used to treat men with asymptomatic or advanced metastatic castrate-resistant prostate cancer. Despite improvement in median overall survival, most patients did not achieve PSA response to therapy. This patient, however, had an improvement of PSA response from 9.29 ng/ml to 6.4 ng/ml following immunotherapy. Interestingly, his EMT-related gene profile in CTCs most closely resembles that of patients with castrate-sensitive disease. One of the limitations of our study is small sample size, however, it is possible that this type of single-cell analysis may have a predictive role in a subset of patients with castrate-resistant disease who would benefit from immunotherapy.

In this regard, we additionally conducted a microfluidics-based PCR analysis of 12 oncogenes for which targeted inhibitors are readily available in early phase clinical studies at our institution. The CTC analysis on these patients will allow their clinicians to consider targeted treatments, such as PIM kinase inhibitors, mTOR inhibitors, G-202 (a PSMA targeting pro-drug), Axl and MUC-1 inhibitors, as therapeutic options for these men with castrate and chemo-resistant disease who have exhausted all FDA-approved agents available to them. For example, three genes—PIM3, MTOR, and ACP5 were frequently found in CTCs of both castration-resistant and -sensitive patients. This finding suggests that metastatic potential of CTCs may depend on the oncogenic addiction of related signal transduction. Consideration should be given to this type of assessment for patients with advanced prostate who have failed hormone ablation and second-line therapies.

### Use of Gene Panel Data to Differentiate Cancer Cell Lines Having Different Metastatic Status

We undertook studies to verify if the EMT gene panel and stem cell and drug target gene panel (Table 2) could differentiate well-established prostate cancer cell lines with different metastatic status.

TABLE 2

Gene Panel I:
EMT
ACTB
ALDH1(A1)
ASPA
BMP7
CD133 (PROM1)
CDH1
CDH2
COL1A2
CXCL13
DAB2IP
EEF1G
EGFR
EpCAM
ESR2 (ESRb)
FN1
FOXC2
FZD4
GAPDH
GSK3B
IGFBP4
ITGA5
ITGB1 (CD29)
KLK4
KRT7(CK7)
KRT8(CK8)
MMP9
NOTCH1
PCA-3
PSA (KLK3)
PTCH1
PTCH2
PTPRC (CD45)
SMAD2
SNAI1
SNAI2
SOX9
TCF3
TGFB1
TGFB2
TGFB3
TWIST1
UBB
VIM
WNT11
WNT5A
WNT5B
ZEB1
ZEB2
Gene Panel II:
Stem cell and drug target
ACP5
ACPP (PSAP)
ACTB
ADRA2A
ALPL (BAP)
ATXN1 (SCA-1)
AURKA
AXL
CD44
CK5
CTNNB1
EEF1G
ERBB2
FOLH1 (PSMA)
FOXA2
FOXP3
FZD7
GAPDH
GATA3
GLI-1
GLI-2
GLI-3
HERPUD1 (MIF1)
IGF1
IGF1R
IGF2
ITGA6
KLK2
MKI67 (Ki-67)
MMP14
MMP2
MMP7
MTOR
MUC1
MYC
NKX3-1
PIM1
PIM2
PIM3
PTEN
PTPRN2
SHH
SPP1
SRC1
TACSTD2
TNFRSF11B
TNFSF11(RANKL)
UBB

The goal is to perform proof-of-principle experiments to verify if EMT gene expression profiles are correlated with metastatic malignancy and aggressiveness using known metastatic prostate cancer. BPH-1 is a benign and non-metastatic prostate cancer cells whereas LNCaP, DU145 and PC3 are metastatic. Among the metastatic cells, DU145 and PC3 are more advanced and aggressive prostate cancer cells showing castration-resistance. Based on our previous study, we hypothesized that EMT genes are expressed differentially in different prostate cancer cells according to their metastatic status. The more advanced metastatic status exhibits higher expression of EMT genes. Single cells (n=48) were collected from these four cells lines and subjected to BioMark™ qRTPCR using the primers from EMT panel and Stem Cell and Drug Target panel. The gene expression profiling was obtained as described previously. The expression data are subsequently subject to hierarchical clustering analysis, violin plot analysis and Ingenuity Pathway Analysis (IPA).

Hierarchical clustering analysis results of single-cell EMT gene expression of prostate cancer cells showed that advanced metastatic cells have higher EMT gene expression compared to benign or less metastatic prostate cancer cells. This indicates that EMT gene expressions are able to classify the cells with differentially distinct metastatic statuses. The metastatic groups have higher EMT gene expression as compared to BPH-1 cells. The in vitro data support our previous study that EMT gene expression profiling is able to separate CTCs from castration-sensitive and castration-resistant prostate cancer. Among three groups of metastatic prostate cancer cells, each cell group has its own heatmap pattern. These group-specific EMT gene profiles can serve as group signatures to further stratify metastatic status among metastatic prostate cancer.

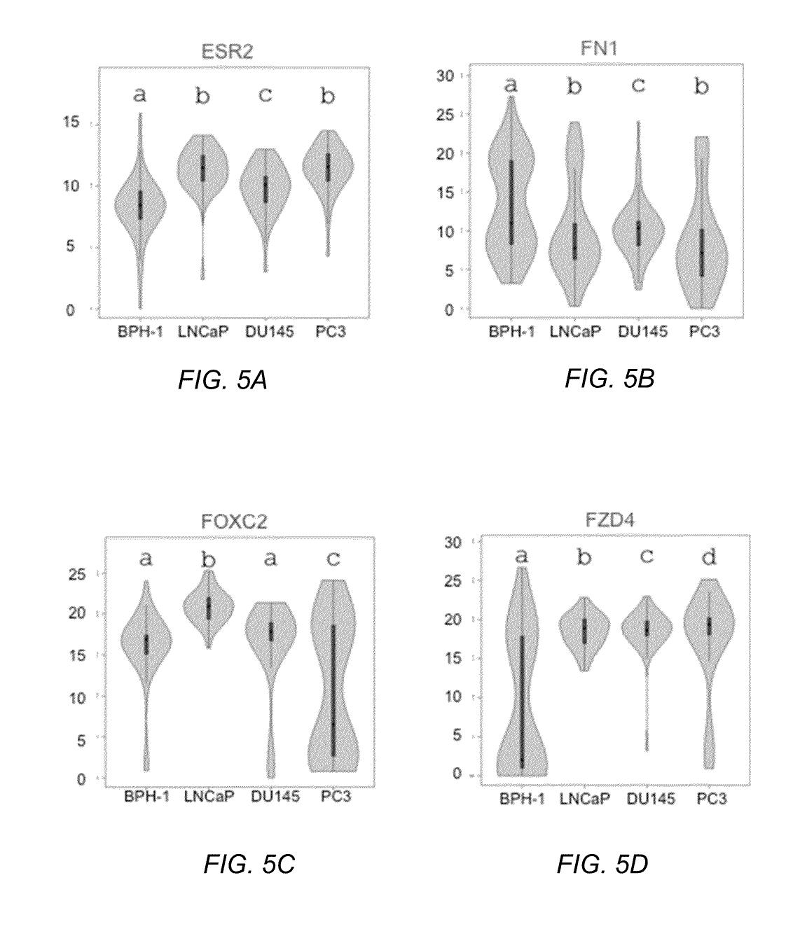

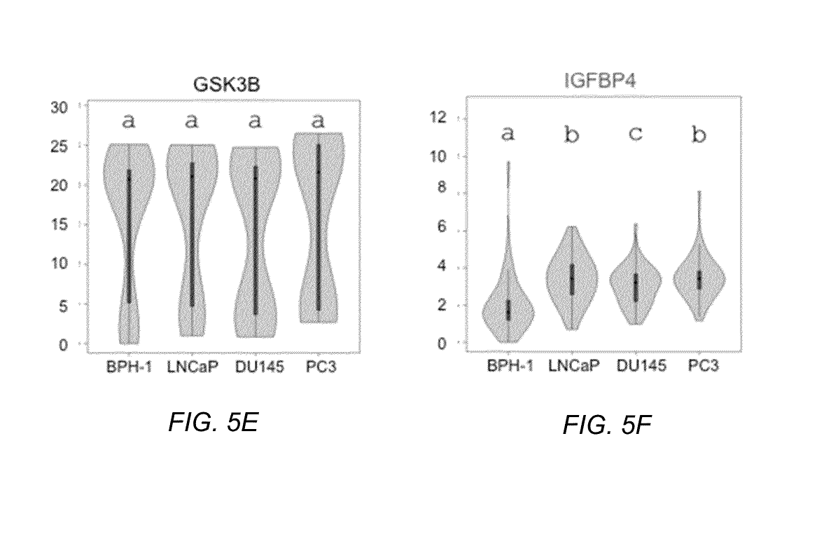

The data were further analyzed using violin plots and IPA to identify specific EMT genes or relevant pathways that may significantly contributed to the metastasis. We hypothesized that the EMT genes with differential expression profiles in each metastatic prostate cancer group would contribute significantly to its metastatic malignancy and could be potential therapeutic targets. FIGS. 5A-5F depict representative violin plots of EMT, drug target, stem gene panel expression profiles in four prostate cancer cells. Expressions are diverse among the EMT genes. Some genes are upregulated in metastatic prostate cancer cells, such as ESR2 (FIG. 5A), FZD4 (FIG. 5D) and IGFBP4 (FIG. 5F), whereas some genes are downregulated, like FN1 (FIG. 5B). GSK3B (FIG. 5E) shows no difference among all the cells. FOXC2 (FIG. 5C) displays diverse expression following no particular metastatic trend. Genes with differential expressions in metastatic prostate cancer cells implicates their roles in metastatic malignancy. Some of genes have FDA-approved target drugs for clinical application. Targeted manipulation of gene expression via whether suppression or activation, may intervene these gene malignant functions and compromise metastatic capacity to spread to distant organs.

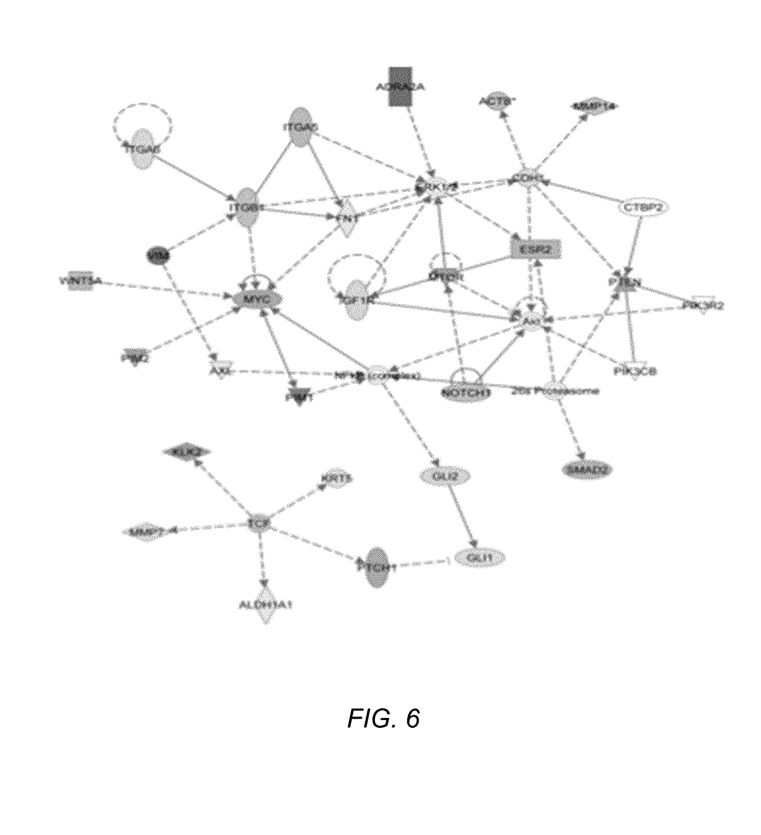

Ingenuity Pathway Analysis (“IPA”) was used to analyze data and discover some major signaling pathways that may contribute to the metastasis and also serve as potential therapeutic targets. FIG. 6 depicts pathways shared by EMT genes upregulated and downregulated in metastatic prostate cancer cells. Several pathways, MYC, ITGB1, ERK1/2, Akt1, NFκB and TCF might be involved in the metastasis of PC3 cells. These pathways could be potential therapeutic targets for metastatic prostate cancer. EMT genes upregulated (red) and downregulated (green) are connected with arrows representing interactions. Arrowheads represent interaction directions. Solid arrows indicate direct interactions whereas dashed arrows indirect interactions.

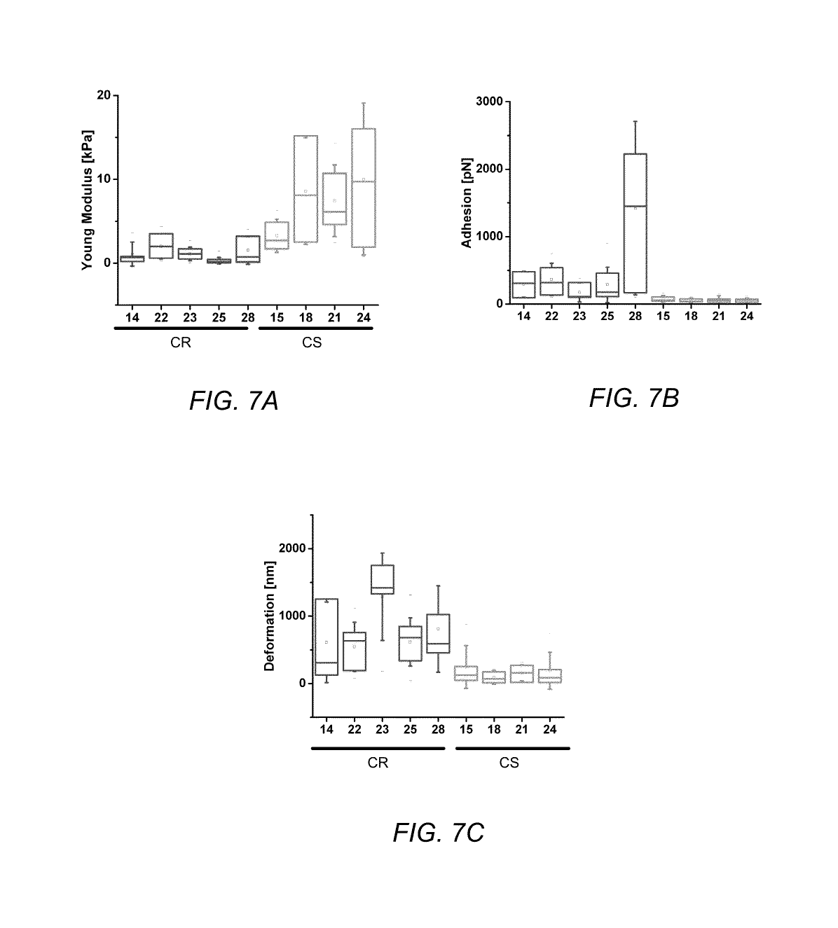

FIGS. 7A-7C depicts box plots of Young modulus, Adhesion and Deformation of CTCs isolated from castration resistant (CR) and sensitive (CS) patients. As can be seen in FIGS. 7A-7C, CTCs have very different nanomechanical properties depending on the type of cancer present. Data was collected with PeakForce QNM Catalyst AFM (Bruker). Boxes represent data obtained from individual patients (red=CR, green=CS). FIG. 7A shows that CTCs of CR patients are about 3 fold more elastic than CTCs of CS patients (expressed as the Young modulus in kPa). Lower values of the Young modulus represent higher cell elasticity. FIG. 7B shows CTCs of CR patients are about 7 fold more adherent than CTCs of CS patients (expressed in units of force pN). Higher values of adhesiveness correspond to more sticky cells. FIG. 7C shows that CTCs of CR patients are about 3 fold more deformable than CTCs of CS patients (expressed as indentation depth in units of length nm). Higher values of deformations are interpreted as a deeper indentation of cells during their poking with an AFM tip. For each patient at least 3 cell were analyzed (maximum n=11 cells). The boxes represent data in a 25-75 range, horizontal lines within the boxes show a median, whiskers are SD, open squares represent arithmetic mean, and small dashes denote maxima and minima. The results of this study are summarized in Table 3.

TABLE 3

| | CR (Mean SD) | CS |
|---|---|---|
| Young Modulus | 1.18 + 1.30 kPa n = 50 | 3.05 + 2.59 n = 23 |
| Adhesion | 466 + 766 pN n = 49 | 61.4 + 41.0 n = 27 |
| Deformation | 731 + 534 nm n = 46 | 259 + 244 n = 19 |

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of analyzing circulating tumor cells comprising:
separating circulating tumor cells from a blood sample by filtration, wherein the circulating tumor cells represent at least a portion of cells retained by a filter of the filtration system;
separating CD45-negative cells from the retained cells on the filter; and
determining the nanomechanical and/or nanochemical properties of the collected CD45-negative cells.

2. The method of claim 1, wherein separating CD45-negative cells from the retained cells comprises:
staining the retained cells with an anti-CD45 antibody coupled to a visualization agent; and
physically separating the unstained circulating tumor cells from the stained cells.

3. The method of claim 2, wherein physically separating the unstained circulating tumor cells from the stained cells comprises collecting the unstained circulating tumor cells using a micromanipulator and placing the collected unstained circulating tumor cells in a container.

4. The method of claim 1, wherein determining the nanomechanical properties of the CD45-negative circulating tumor cells comprises determining the elasticity of the CD-45 negative circulating tumor cells.

5. The method of claim 4, wherein determining the elasticity of the CD-45 negative circulating tumor cells comprises applying an atomic force microscope probe to at least one CD-45 negative circulating tumor cell and creating a cellular indentation mapping of the CD45-negative circulating tumor cell.

6. The method of claim 1, wherein determining the nanomechanical properties of the CD45-negative circulating tumor cells comprises determining the smoothness of the collected CD45-negative circulating tumor cells.

7. The method of claim 6, wherein determining the smoothness of the collected CD45-negative circulating tumor cells comprises collecting one or more images of the collected CD45-negative circulating tumor cells and determining an average variance in height of the collected CD45-negative circulating tumor cells.

8. The method of claim 1, wherein determining the chemical features of the CD45-negative circulating tumor cells comprises determining the adhesiveness of the collected CD45-negative circulating tumor cells.

9. The method of claim 8, wherein determining the adhesiveness of the collected CD45-negative circulating tumor cells comprises determining the force needed to pull a tip out of contact from a collected CD45-negative circulating tumor cell.

10. The method of claim 1, further comprising lysing the collected CD45-negative circulating tumor cells and performing RT-PCR analysis of the lysed CD45-negative circulating tumor cells to determine the presence of metastatic specific genes.

11. A method of identifying metastatic behavior of circulating tumor cells comprising:
separating circulating tumor cells from a blood sample by filtration, wherein the circulating tumor cells represent at least a portion of cells retained by a filter of the filtration system;
separating CD45-negative cells from the retained circulating tumor cells and blood cells;
determining the nanomechanical and/or nanochemical properties of the collected CD45-negative cells; and
determining the metastatic behavior of the collected CD45-negative cells based on the nanomechanical and/or nanochemical properties of the collected CD45-negative cells.

12. The method of claim 11, wherein separating CD45-negative cells from the collected circulating tumor cells comprises:
staining the retained cells with an anti-CD45 antibody coupled to a visualization agent; and
physically separating the unstained circulating tumor cells from the stained cells.

13. The method of claim 12, wherein physically separating the unstained circulating tumor cells from the stained cells comprises collecting the unstained circulating tumor cells using a micromanipulator and placing the collected unstained circulating tumor cells in a container.

14. The method of claim 11, wherein determining the nanomechanical properties of the CD45-negative circulating tumor cells comprises determining the elasticity of the CD-45 negative circulating tumor cells.

15. The method of claim 14, wherein determining the elasticity of the CD-45 negative circulating tumor cells comprises applying an atomic force microscope probe to at least one CD-45 negative circulating tumor cell and creating a cellular indentation mapping of the CD45-negative circulating tumor cell.

16. The method of claim 11, wherein determining the nanomechanical properties of the CD45-negative circulating tumor cells comprises determining the smoothness of the collected CD45-negative circulating tumor cells.

17. The method of claim 16, wherein determining the smoothness of the collected CD45-negative circulating tumor cells comprises collecting one or more images of the collected CD45-negative circulating tumor cells and determining an average variance in height of the collected CD45-negative circulating tumor cells.

18. The method of claim 11, wherein determining the nanochemical properties of the CD45-negative circulating tumor cells comprises determining the adhesiveness of the collected CD45-negative circulating tumor cells.

19. The method of claim 18, wherein determining the adhesiveness of the collected CD45-negative circulating tumor cells comprises determining the force needed to pull a tip out of contact from a collected CD45-negative circulating tumor cell.

20. The method of claim 11, further comprising lysing the collected CD45-negative circulating tumor cells and performing microfluidic single-cell RT-PCR analysis of the lysed CD45-negative circulating tumor cells to determine the expression profile of metastatic specific genes.
